(19) United States
(12) Patent Application Publication
(10) Pub. No.: US 2020/0232079 A1
(43) Pub. Date: Jul. 23, 2020

(54) FE-MN ABSORBABLE IMPLANT ALLOYS WITH INCREASED DEGRADATION RATE

(71) Applicant: Bio DG, Inc., Poway, CA (US)

(72) Inventor: John A. DISEGI, Poway, CA (US)

(21) Appl. No.: 16/095,104

(22) PCT Filed: Oct. 5, 2018
(86) PCT No.: PCT/US2018/054686

§ 371 Date: Oct. 19, 2018

(51) Int. Cl. C22C 38/60; C22C 38/04; C22C 33/06; C22C 33/02; A61C 8/00; A61F 2/28; A61B 17/064; A61B 17/86; A61F 2/00; A61L 31/14; A61L 31/02; A61L 27/04; A61L 27/58

(52) U.S. Cl. CPC C22C 38/60; A61F 2002/0876; C22C 33/06; C22C 33/0221; C22C 33/0278; A61C 8/0012; A61F 2/2875; A61B 17/064; A61B 17/86; A61F 2/0063; A61L 31/148; A61L 31/022; A61L 27/047; A61L 27/58; A61L 27/042; C22C 38/04

(57) ABSTRACT

The present invention is directed to a biodegradable alloy suitable for use in a medical implant, comprising at least 50% iron by weight, at least 25% manganese by weight, and at least 0.01% sulfur and/or selenium by weight, wherein the biodegradable alloy is nonmagnetic. The present invention also provides a method of producing a biodegradable alloy with a desirable degradation rate.

### CROSS-REFERENCE TO RELATED APPLICATIONS

This application claims the benefit of and priority to U.S. Provisional Application No. 62/569,228, filed on Oct. 6, 2017, the contents of which are hereby incorporated by reference.

### FIELD OF THE INVENTION

The present invention relates to biodegradable Fe—Mn alloys.

### BACKGROUND OF THE INVENTION

Iron, magnesium, or zinc based metals with or without other alloying elements have been evaluated for the manufacture of absorbable metallic implants. Absorbable metallic implants are designed to degrade in the body as a result of corrosion reactions which occur over a period of time. The degradation products should be transported and eliminated without local or systemic accumulation in the body. The implant degradation rate must be balanced against the level of mechanical integrity that is required to achieve functionality over a specified timeframe.

Absorbable Fe—Mn alloys have been extensively researched over the years for cardiovascular applications. Studies by Liu and Zheng (Acta Biomater., 7, 1407-1420 (2011)) investigated binary alloy FeS degradation rates close to that of pure iron which indicated no improvement compared to Fe—Mn alloys. The FeS binary alloys investigated by Liu and Zheng did not contain Mn. Other research has determined that a minimum 25% manganese addition is required to provide a completely nonmagnetic microstructure (Hermawan, H., Metallic Biodegradable Coronary Stent: Materials Development in Biodegradable Metals From Concept to Applications, Chapter 4, Springer, 43-44 (2012)). A nonmagnetic implant microstructure is necessary to allow patient exposure to magnetic resonance imaging (MRI) procedures.

Lightweight cardiovascular stents are usually fabricated from seamless tubing which is machined or laser cut to include intricate tubular wall patterns. The outside diameters of stents are typically <2.0 mm and are usually inserted into a small or large artery by a catheter (Hermawan, H., Biodegradable Metals for Cardiovascular Applications, in Biodegradable Metals from Concept to Applications, Chapter 3, Springer, 23-24 (2012)). However, the degradation rate of Fe—Mn absorbable alloys is too slow for moderate sized metallic medical implants such as plates, screws, nails, bone anchors, etc. Moderate sized medical implants are defined as implants that exceed the mass of cardiovascular or neurological stents.

Therefore, there is a need for biodegradable Fe—Mn alloys with desirable degradable rates.

### SUMMARY OF THE INVENTION

One aspect of the invention relates to a biodegradable alloy suitable for use in a medical implant, comprising at least 50% iron by weight, at least 25% manganese by weight, and at least 0.01% sulfur and/or selenium by weight, wherein the biodegradable alloy is nonmagnetic.

In some embodiments, the biodegradable alloy is substantially free of chromium.

In some embodiments, the biodegradable alloy is substantially free of nickel.

In some embodiments, sulfur and manganese form a manganese sulfide secondary phase.

In some embodiments, selenium and manganese form a manganese selenide secondary phase.

In some embodiments, the sulfur or selenium is dispersed equally in the biodegradable alloy.

In some embodiments, the biodegradable alloy comprises at least 60% iron by weight.

In some embodiments, the biodegradable alloy comprises at least 30% manganese by weight.

In some embodiments, the biodegradable alloy is in the form of a wrought product, a cast product, or a powder metallurgy product.

In some embodiments, the biodegradable alloy has a degradation rate of about 0.155 to 3.1 mg/cm² under physiological conditions.

In some embodiments, the biodegradable alloy comprises 0.01% to 0.35% sulfur and/or selenium by weight.

In some embodiments, the biodegradable alloy comprises 0.01% to 0.20% sulfur and/or selenium by weight.

In some embodiments, the biodegradable alloy comprises 0.02% to 0.10% sulfur and/or selenium by weight.

Another aspect of the invention relates to an implantable medical device comprising a biodegradable alloy disclosed herein. In some embodiments, the implantable medical device is selected from the group consisting of a bone screw, a bone anchor, a tissue staple, a craniomaxillofacial reconstruction plate, a surgical mesh, a fastener (e.g., a surgical fastener), a reconstructive dental implant, and a stent.

Another aspect of the invention relates to a method of producing a biodegradable alloy with a desirable degradation rate, the method comprising: (a) adding a composition comprising sulfur and/or selenium to a molten mixture to produce the biodegradable alloy, wherein the molten mixture has at least 50% iron by weight and at least 25% manganese by weight, and wherein the biodegradable alloy comprises at least 0.01% sulfur and/or selenium by weight, and (b) cooling the biodegradable alloy.

In some embodiments, the biodegradable alloy is substantially free of chromium.

In some embodiments, the biodegradable alloy is substantially free of nickel.

In some embodiments, the sulfur and/or selenium is added at 100 to 3500 parts per million.

In some embodiments, the composition comprising sulfur is iron(II) sulfide.

In some embodiments, the composition comprising selenium is iron(II) selenide.

In some embodiments, the sulfur or selenium is dispersed equally in the biodegradable alloy.

In some embodiments, the biodegradable alloy comprises at least 60% iron by weight.

In some embodiments, the biodegradable alloy comprises at least 30% manganese by weight.

In some embodiments, the molten mixture is substantially free of silicon.

In some embodiments, the molten mixture is substantially free of aluminum.

In some embodiments, the molten mixture is substantially free of oxygen.

In some embodiments, the method further comprises adding a basic slag to the molten mixture, thereby removing oxygen from the molten mixture to the basic slag. In some embodiments, the basic slag comprises a calcium oxide to silicon dioxide ratio of at least 2.

In some embodiments, the biodegradable alloy is cooled at a rate of 30° C./min to 60° C./min.

In some embodiments, the biodegradable alloy comprises 0.01% to 0.35% sulfur and/or selenium by weight.

In some embodiments, the biodegradable alloy comprises 0.01% to 0.20% sulfur and/or selenium by weight.

In some embodiments, the biodegradable alloy comprises 0.02% to 0.10% sulfur and/or selenium by weight.

Yet another aspect of the invention relates to a method of producing a biodegradable alloy with a desirable degradation rate, the method comprising adding 100 to 3500 parts per million sulfur to a molten mixture having at least 50% iron by weight and at least 25% manganese by weight, thereby producing a biodegradable alloy having at least 0.01% sulfur by weight.

### BRIEF DESCRIPTION OF THE DRAWINGS

FIG. 1 is a schematic depicting the elongated MnS secondary phase in wrought product form.

FIG. 2 is a schematic depicting the globular MnS secondary phase in cast or powder product form.

### DETAILED DESCRIPTION OF THE INVENTION

The present invention is based, inter alia, on the discovery that the formation of manganese sulfide precipitates in steels has been shown to increase corrosion rates. Manganese (II) sulfide (MnS) precipitates have also been shown to be more chemically active than the surrounding steel alloy. In some embodiments, as the Fe—Mn steel is cold worked by drawing into elongated forms, such as bars, tubing, or wires, the MnS precipitates fracture and leave voids within the form, thereby creating additional corrosion surfaces. Corrosion is the primary degradation mechanism for biodegradable implants and increased corrosion rates equate to faster degradation profiles for biodegradable implants.

It is the objective of this invention to increase the degradation rate of absorbable Fe—Mn alloys by adding sulfur (S) or selenium (Se) to the alloy. In some embodiments, the amount of intentionally added sulfur or selenium in the Fe—Mn alloy can be similar to the amount of sulfur or selenium added to free-machining stainless steel. For example, the relative amount of sulfur or selenium in free-machining non-implantable Type 303 stainless steels, non-absorbable implant quality Type 316L, and Fe—Mn absorbable implant alloy as disclosed herein, are shown in Table 1.

TABLE 1
Sulfur and Selenium Content of Alloys

| Alloy | Sulfur | Selenium | Implant- | Absorb- |
|---|---|---|---|---|
| Type | Content (%) | Content (%) | able | able |
| Free-Machining | Min 0.150 | N/A | No | No |
| 303 | | | | |
| Free-Machining | N/A | 0.15-0.35 | No | No |
| 303 Se | | | | |
| 316L Implant | Max 0.010 | N/A | Yes | No |
| Quality | | | | |
| Fe—Mn Implant | 0.01-0.35 | N/A | Yes | Yes |
| Quality | | | | |
| Fe—Mn Implant | N/A | 0.01-0.35 | Yes | Yes |
| Quality | | | | |

In one aspect, the present disclosure provides a biodegradable alloy suitable for use in a medical implant, comprising at least 50% iron by weight, at least 25% manganese by weight, and at least 0.01% sulfur and/or selenium by weight, wherein the biodegradable alloy is nonmagnetic. The sulfur or selenium can be dispersed equally in the biodegradable alloy.

The biodegradable alloy may or may not contain minor additions of carbon, nitrogen, phosphorous, silicon, or trace elements typically associated with Fe—Mn alloys. In some embodiments, the biodegradable alloy is substantially free of chromium. In some embodiments, the biodegradable alloy is substantially free of nickel. As used herein, the term “substantially free” when referring to the presence of an element in a biodegradable alloy means that the concentration of the element in the biodegradable alloy is no more than 0.2%, no more than 0.1%, or no more than 0.05% by weight.

In some embodiments, the biodegradable alloy includes at least 55% iron by weight, e.g., at least 60% iron by weight, at least 65% iron by weight, or at least 70% iron by weight. In some embodiments, the biodegradable alloy includes 50% to 70% iron by weight, e.g., 50% to 60% iron by weight, 55% to 60% iron by weight, 55% to 70% iron by weight, or 60% to 70% iron by weight.

In some embodiments, the biodegradable alloy includes at least 28% manganese by weight, e.g., at least 30% manganese by weight, at least 35% manganese by weight, at least 40% manganese by weight, or at least 45% manganese by weight. In some embodiments, the biodegradable alloy includes 25% to 45% manganese by weight, e.g., 25% to 40% manganese by weight, 25% to 35% manganese by weight, 30% to 45% manganese by weight, or 35% to 45% manganese by weight.

In some embodiments, the biodegradable alloy includes 0.01% to 2.0% sulfur by weight. In some embodiments, the biodegradable alloy includes 0.01% to 1.5% sulfur by weight. In some embodiments, the biodegradable alloy includes 0.01% to 1.2% sulfur by weight. In some embodiments, the biodegradable alloy includes 0.01% to 1.0% sulfur by weight. In some embodiments, the biodegradable alloy includes 0.01% to 0.35% sulfur by weight. In some embodiments, the biodegradable alloy includes 0.01% to 0.30% sulfur by weight. In some embodiments, the biodegradable alloy includes 0.01% to 0.20% sulfur by weight. In some embodiments, the biodegradable alloy includes 0.01% to 0.15% sulfur by weight. In some embodiments, the biodegradable alloy includes 0.02% to 0.10% sulfur by weight. In some embodiments, the biodegradable alloy includes 0.10% to 0.35% sulfur by weight. In some embodiments, the biodegradable alloy includes 0.15% to 0.35% sulfur by weight. In some embodiments, the biodegradable alloy includes 0.20% to 0.35% sulfur by weight. In some embodiments, the biodegradable alloy includes 0.5% to 2.0% sulfur by weight. In some embodiments, the biodegradable alloy includes 0.5% to 1.5% sulfur by weight. In some embodiments, the biodegradable alloy includes 0.5% to 1.2% sulfur by weight. In some embodiments, the biodegradable alloy includes 0.5% to 1.0% sulfur by weight.

In some embodiments, the biodegradable alloy includes 0.01% to 2.0% selenium by weight. In some embodiments, the biodegradable alloy includes 0.01% to 1.5% selenium by weight. In some embodiments, the biodegradable alloy includes 0.01% to 1.2% selenium by weight. In some embodiments, the biodegradable alloy includes 0.01% to 1.0% selenium by weight. In some embodiments, the biodegradable alloy includes 0.01% to 0.35% selenium by weight. In some embodiments, the biodegradable alloy includes 0.01% to 0.30% selenium by weight. In some embodiments, the biodegradable alloy includes 0.01% to 0.20% selenium by weight. In some embodiments, the biodegradable alloy includes 0.01% to 0.15% selenium by weight. In some embodiments, the biodegradable alloy includes 0.02% to 0.10% selenium by weight. In some embodiments, the biodegradable alloy includes 0.10% to 0.35% selenium by weight. In some embodiments, the biodegradable alloy includes 0.15% to 0.35% selenium by weight. In some embodiments, the biodegradable alloy includes 0.20% to 0.35% selenium by weight. In some embodiments, the biodegradable alloy includes 0.5% to 2.0% selenium by weight. In some embodiments, the biodegradable alloy includes 0.5% to 1.5% selenium by weight. In some embodiments, the biodegradable alloy includes 0.5% to 1.2% selenium by weight. In some embodiments, the biodegradable alloy includes 0.5% to 1.0% selenium by weight.

In some embodiments, the biodegradable alloy includes 0.01% to 2.0% sulfur and selenium by weight. In some embodiments, the biodegradable alloy includes 0.01% to 1.5% sulfur and selenium by weight. In some embodiments, the biodegradable alloy includes 0.01% to 1.2% sulfur and selenium by weight. In some embodiments, the biodegradable alloy includes 0.01% to 1.0% sulfur and selenium by weight. In some embodiments, the biodegradable alloy includes 0.01% to 0.35% sulfur and selenium by weight. In some embodiments, the biodegradable alloy includes 0.01% to 0.30% sulfur and selenium by weight. In some embodiments, the biodegradable alloy includes 0.01% to 0.20% sulfur and selenium by weight. In some embodiments, the biodegradable alloy includes 0.01% to 0.15% sulfur and selenium by weight. In some embodiments, the biodegradable alloy includes 0.02% to 0.10% sulfur and selenium by weight. In some embodiments, the biodegradable alloy includes 0.10% to 0.35% sulfur and selenium by weight. In some embodiments, the biodegradable alloy includes 0.15% to 0.35% sulfur and selenium by weight. In some embodiments, the biodegradable alloy includes 0.20% to 0.35% sulfur and selenium by weight. In some embodiments, the biodegradable alloy includes 0.5% to 2.0% sulfur and selenium by weight. In some embodiments, the biodegradable alloy includes 0.5% to 1.5% sulfur and selenium by weight. In some embodiments, the biodegradable alloy includes 0.5% to 1.2% sulfur and selenium by weight. In some embodiments, the biodegradable alloy includes 0.5% to 1.0% sulfur and selenium by weight. The weight ratio of sulfur to selenium can be in the range of 99:1 to 1:99. For example, the weight ratio of sulfur to selenium can be in the range of 99:1 to 75:1, 99:1 to 50:1, or 90:1 to 50:1.

In some embodiments, the biodegradable alloy includes 50% to 70% iron by weight, 25% to 35% manganese by weight, and 0.01% to 0.35% sulfur by weight.

In some embodiments, the biodegradable alloy includes 50% to 70% iron by weight, 25% to 35% manganese by weight, and 0.01% to 0.35% selenium by weight.

In some embodiments, the biodegradable alloy includes 50% to 70% iron by weight, 25% to 35% manganese by weight, and 0.01% to 0.35% sulfur and selenium by weight. The weight ratio of sulfur to selenium can be in the range of 1:99 to 99:1. For example, the weight ratio of sulfur to selenium can be in the range of 99:1 to 75:1, 99:1 to 50:1, or 90:1 to 50:1.

Depending on the concentration of sulfur and/or selenium, the degradation rate of the biodegradable alloy can be in the rage of about 0.155 to 3.1 mg/cm² per day under physiological conditions. In some embodiments, the degradation rate of the biodegradable alloy can be in the rage of about 0.2 to 3.0 mg/cm² per day under physiological conditions. In some embodiments, the degradation rate of the biodegradable alloy can be in the rage of about 0.2 to 2.5 mg/cm² per day under physiological conditions. In some embodiments, the degradation rate of the biodegradable alloy can be in the rage of about 1.0 to 3.1 mg/cm² per day under physiological conditions. The degradation rate of the biodegradable alloy can also be at least 0.3 mg/cm² per day, at least 0.4 mg/cm² per day, at least 0.5 mg/cm² per day, at least 1.0 mg/cm² per day, at least 1.5 mg/cm² per day, at least 2.0 mg/cm² per day, or at least 2.5 mg/cm² per day.

In some embodiments, the term “physiological conditions” refers to a temperature range of 20-40° C., atmospheric pressure of 1, pH of 6-8, glucose concentration of 1-20 mM, atmospheric oxygen concentration, and earth gravity. Thus, the present disclosure provides a series of fully or partially densified Fe—Mn alloys with controlled sulfur or selenium content in order to establish a defined range of implant degradation rates. Small and moderate size Fe—Mn absorbable implants with improved machinability and predictable degradation rates can be designed depending on the application.

The biodegradable alloy can be in the form of a wrought product, a cast product, or a powder metallurgy product.

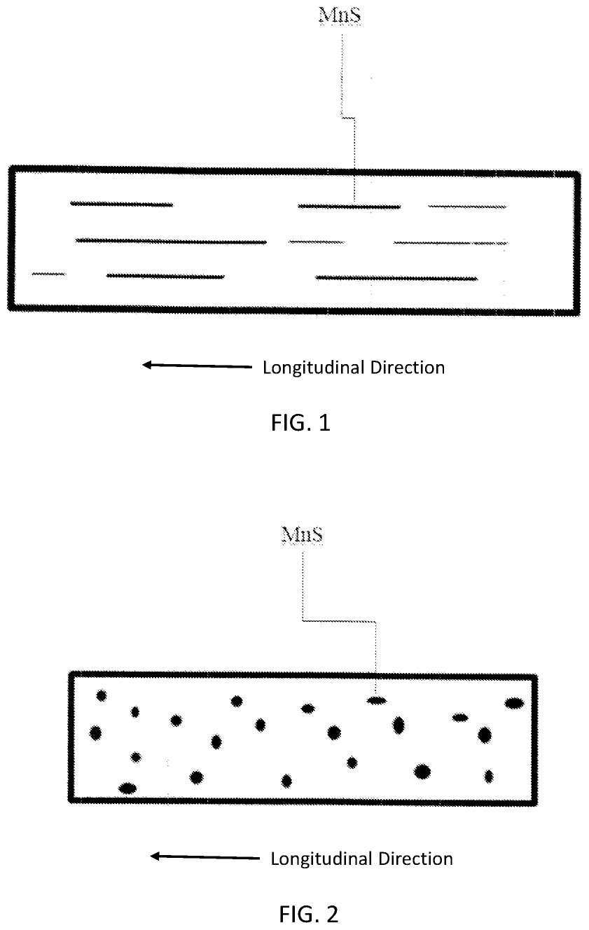

Sulfur additions to Fe—Mn alloys form a MnS secondary phase in the microstructure. Similarly, selenium addition to Fe—Mn alloys form a MnSe secondary phase in the microstructure. Wrought Fe—Mn alloys containing a MnX (X═S or Se) secondary phase may be processed to semi-finished product forms by wrought hot, warm, or ambient temperature metalworking operations such as, but not limited to, pressing, forging, rolling, extrusion, swaging, and drawing. All of these wrought metalworking operations reduce the cross-sectional area and create an elongated MnX secondary phase known as a stringer in the longitudinal direction. The elongated MnX stringer morphology is depicted in FIG. 1. The MnX secondary phase provides enhanced machinability and increased pitting and crevice corrosion reactions in a multitude of chemical solutions when compared to the corrosion rate of the bulk matrix. Wrought product forms may be processed and machined into Fe—Mn absorbable medical devices depending on the implant application. Depending on the application, the wrought semi-finished product form may be machined, cleaned, passivated, sterilized, and packaged to produce a finished implant device.

Investment casting can be used to produce Fe—Mn cast shapes with a MnX secondary phase. Castings may contain internal imperfections, large grain size, and chemical segregation, which typically can have a deleterious effect on mechanical properties and magnetic response. Secondary operations such as hot isostatic pressing can be used to improve as-cast properties. When compared to casting technology, wrought metalworking practices previously described are capable of providing fewer internal imperfections, smaller grain size, and improved mechanical properties.

Specialty melted or conventionally melted Fe—Mn absorbable alloy bar or billet containing sulfur additions may be used as starting stock, known as an electrode, to produce a powder metallurgy alloy. The electrode surface is usually conditioned by peeling, centerless grinding, polishing, or other metal removal processes for the elimination of superficial imperfections. Water atomization, argon or helium gas atomization, plasma rotating electrode process (PREP), or other powder manufacturing methods may be used to produce the Fe—Mn alloyed powder. A powder metallurgy manufacturing route can be used for Fe—Mn powder particles that may be consolidated into a simple shape, near-net shape, or net shape by metal injection molding (MIM), cold isostatic pressing, hot isostatic pressing, or other well-known powder consolidation techniques. As used herein, the term “simple shape” refers to a product form that requires extensive machining to meet a finish part drawing. As used herein, the term “near net shape” refers to a semi-finished product form that requires a moderate amount of machining to meet a finish part drawing. As used herein, the term “net shape” refers to a semi-finished product form that requires a minimal amount of machining to meet a finish part drawing. Powder consolidation parameters can be adjusted to provide a fully densified or partially densified semi-finished product form depending on the application. The powder consolidated semi-finished product form may be finish machined, cleaned, passivated, sterilized (optional), and packaged to produce a finished implant device.

The major advantage is that a powder metallurgy absorbable implant device contains a fine globular MnX secondary phase as a result of the small powder particle size and the powder processing steps. This avoids the typical stringer or elongated MnX morphology that is associated with wrought metalworking operations. Powder metallurgical methods are capable of providing a consolidated powder product that demonstrates a fine-grained globular MnX morphology, which facilitates good machinability and predictable corrosion response. FIG. 2 is an illustration of the globular MnX morphology in a powder metallurgical product form.

As persons skilled in the art will readily recognize, there are a wide array of implantable medical devices that can be made using the alloys disclosed herein. The biodegradable alloy can be used to produce implantable medical devices that include, but are not limited to, a bone screw, a bone anchor, a tissue staple, a craniomaxillofacial reconstruction plate, a surgical mesh, a fastener (e.g., a surgical fastener), a reconstructive dental implant, or a stent. In certain embodiments, the implantable medical device is a bone anchor (e.g., for the repair of separated bone segments). In other embodiments, the implantable medical device is a bone screw (e.g., for fastening fractured bone segments). In other embodiments, the implantable medical device is a bone immobilization device (e.g., for large bones). In other embodiments, the implantable medical device is a staple for fastening tissue. In other embodiments, the implantable medical device is a craniomaxillofacial reconstruction plate or fastener. In other embodiments, the implantable medical device is a surgical mesh. In other embodiments, the implantable medical device is a dental implant (e.g., a reconstructive dental implant). In still other embodiments, the implantable medical device is a stent (e.g., for maintaining the lumen of an opening in an organ of an animal body).

In some embodiments, the implantable medical device is designed for implantation into a human. In other embodiments, the implantable medical device is designed for implantation into a pet (e.g., a dog, a cat). In other embodiments, the implantable medical device is designed for implantation into a farm animal (e.g., a cow, a horse, a sheep, a pig, etc.). In still other embodiments, the implantable medical device is designed for implantation into a zoo animal.

It is frequently desirable to incorporate bioactive agents (e.g., drugs) on implantable medical devices. For example, U.S. Pat. No. 6,649,631 claims a drug for the promotion of bone growth which can be used with orthopedic implants. Bioactive agents may be incorporated directly on the surface of an implantable medical device of the invention. For example, the agents can be mixed with a polymeric coating, such as a hydrogel of U.S. Pat. No. 6,368,356, and the polymeric coating can be applied to the surface of the device. Alternatively, the bioactive agents can be loaded into cavities or pores in the medical devices which act as depots such that the agents are slowly released over time. The pores can be on the surface of the medical devices, allowing for relatively quick release of the drugs, or part of the gross structure of the alloy used to make the medical device, such that bioactive agents are released gradually during most or all of the useful life of the device. The bioactive agents can be, e.g., peptides, nucleic acids, hormones, chemical drugs, or other biological agents, useful for enhancing the healing process.

In one aspect, the present disclosure provides a container containing an implantable medical device of the invention. In some embodiments, the container is a packaging container, such as a box (e.g., a box for storing, selling, or shipping the device). In some embodiments, the container further comprises an instruction (e.g., for using the implantable medical device for a medical procedure).

In another aspect, the present disclosure provides a method of producing a biodegradable alloy with a desirable degradation rate, the method comprising: (a) adding a composition comprising sulfur and/or selenium to a molten mixture to produce the biodegradable alloy, wherein the molten mixture has at least 50% iron by weight and at least 25% manganese by weight, and wherein the biodegradable alloy comprises at least 0.01% sulfur and/or selenium by weight, and (b) cooling the biodegradable alloy.

The degradation rate of the biodegradable alloy can be controlled by changing the concentration of sulfur and/or selenium in the biodegradable alloy. The higher the concentration of sulfur and/or selenium, the faster the degradation rate. In some embodiments, the sulfur and/or selenium is added at 100 to 6000 parts per million (ppm). For example, the sulfur and/or selenium can be added at 300 to 3000 ppm.

In some embodiments, the composition comprising sulfur is S, iron(II) sulfide, FeS₂, Fe₂S₃, or MnS. In some embodiments, the composition comprising selenium is Se, iron(II) selenide, FeSe₂, Fe₂Se₃, or MnSe.

The degradation rate of the biodegradable alloy can also be controlled by changing the size, shape, and/or dispersion of MnX inclusions. Finer, more diffuse inclusions will result in more uniform and faster degradation. Whereas larger inclusions will result in a slower and less uniform corrosion. Both of these conditions may be appropriate, depending on the purpose of the implanted device. Therefore, control over inclusion size is desirable to maximize the versatility of an absorbable alloy. MnX inclusions can also take multiple morphologies from spherical or globular to rod like and angular. In some embodiments, the MnX inclusions have a globular morphology. Spherical/globular MnX inclusions give dispersed, uniform degradation. Angular or elongated MnX inclusions can have more surface area and faster degradation but they can cause early implant failure due to irregular degradation. Therefore, spherical/globular MnX inclusions are more desirable in some applications.

The degradation rate of the biodegradable alloy can also be controlled by controlling the concentration of dissolved oxygen in a steel melt prior to the formation of the biodegradable alloy. Lower levels of dissolved oxygen in a steel melt leads to a more globular MnX shape. In some embodiments, globular inclusions will form at less than 150 ppm dissolved oxygen in the steel melt. In some embodiments, the molten mixture is substantially free of oxygen.

The degradation rate of the biodegradable alloy can also be controlled by controlling the addition of aluminum to a steel melt prior to the formation of the biodegradable alloy. Aluminum affects the shape of the inclusion. The addition of aluminum to a steel melt causes MnX inclusion to become longer, more angular and more easily deformable during subsequent processing. Higher aluminum concentration creates larger, more irregular inclusions. In some embodiments, the molten mixture is the molten mixture is substantially free of aluminum.

The degradation rate of the biodegradable alloy can also be controlled by controlling the concentration of silicon in the biodegradable alloy. Increased silicon concentration increases the length to width ratio of MnX inclusions, thereby increasing the surface area and the degradation rate in a more irregular way. In some embodiments, the molten mixture is substantially free of silicon. In some embodiments, a steel alloy of low silicon, low oxygen, and low aluminum can produce globular inclusions of approximately 1 micron to 20 microns in diameter, e.g., 1 micron to 15 microns in diameter, 1 micron to 10 microns in diameter, or 4 micron to 10 microns in diameter. In some embodiments, a steel alloy of low silicon, low oxygen, and low aluminum can produce globular inclusions of approximately 1 micron in diameter, 2 microns in diameter, 3 microns in diameter, 4 microns in diameter, or 5 microns in diameter.

The degradation rate of the biodegradable alloy can also be controlled by controlling the melt cooling time. Melt cooling times also have an effect on the size and morphology of MnX inclusions. A rapidly cooled melt results in smaller and more dispersed, globular MnX inclusions. The cooling rate for making the biodegradable alloy is dependent on melt temperature, soak time, and ingot size, which will vary depending on the melting method that is employed. In some embodiments, the biodegradable alloy can be cooled at a rate of 10° C./min to 60° C./min, e.g., 10° C./min to 60° C./min, 20° C./min to 60° C./min, 20° C./min to 50° C./min, or 30° C./min to 50° C./min. Cooling after hot working can be much faster than 60° C./min by quenching in water.

The concentrations of aluminum, silicon, and oxygen can be controlled in steel melts by techniques known in the art. The concentrations of aluminum and silicon can be controlled by controlling the quality of the raw materials and the composition of slags used during subsequent electro-slag re-melt (ESR) processing. Primary melting of the alloy in an induction furnace under vacuum or inert gas reduces the levels of atmospheric gases dissolved in the melt. Oxygen can be removed from the melt and into the slag with a highly basic slags, containing a calcium oxide (CaO) to silicon di-oxide (SiO₂) ratio of at least two, and with a very low aluminum oxide (Al₂O₃) to CaO ratio.

Yet another aspect of the invention relates to a method of producing a biodegradable alloy with a desirable degradation rate, the method comprising adding 100 to 3500 parts per million sulfur to a molten mixture having at least 50% iron by weight and at least 25% manganese by weight, thereby producing a biodegradable alloy having at least 0.01% sulfur by weight.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Definitions

The term “comprising” as used herein is synonymous with “including” or “containing” and is inclusive or open-ended and does not exclude additional, unrecited members, elements or method steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “about” means within ±10% of a given value or range.

As used herein, the terms “biodegradable,” “bioabsorbable,” and “bioresorbable” all refer to a material that is able to be chemically broken down in a physiological environment, i.e., within the body or inside body tissue, such as by biological processes including resorption and absorption. This process of chemical breakdown will generally result in the complete degradation of the material and/or appliance within a period of weeks to months, such as 18 months or less, 24 months or less, or 36 months or less, for example. This rate stands in contrast to more “degradation-resistant” or permanent materials and/or appliances, such as those constructed from nickel-titanium alloys (“Ni—Ti”) or stainless steel, which remain in the body, structurally intact, for a period exceeding at least 36 months and potentially throughout the lifespan of the recipient. Biodegradable metals used herein include nutrient metals, e.g., metals such as iron and manganese. These nutrient metals and metal alloys have biological utility in mammalian bodies and are used by, or taken up in, biological pathways.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

A Fe—Mn alloy containing 28.3% manganese, 0.08% carbon, 0.0006% nitrogen, <0.01% silicon, <0.005% phosphorous, 0.0057% sulfur, and balance iron was melted in a vacuum induction furnace into an electrode for secondary melting in an electroslag remelting (ESR) furnace. A sulfur content of 0.0012% was measured after ESR. The resulting ingot was upset forged and hot rolled to an intermediate size and cold rolled to a thickness of 0.094 inch thick. The wrought product form contained an elongated MnS secondary phase when the microstructure was examined in the longitudinal orientation.

### Example 2

An Fe-28 Mn composition containing greater than >0.15% sulfur was vacuum induction melted and cast into a ceramic investment mold containing multiple shaped cavities. After solidification, the ceramic casting shell was removed, castings were cleaned by grit blasting, and the castings were hot isostatic pressed to eliminate internal porosity. The castings contained a globular MnS secondary phase when the microstructure was examined in both the transverse and longitudinal orientation.

### Example 3

A quantity of Fe-28Mn alloy from Example 1 was induction melted and transferred to a water atomizer for the production of irregular metal powder. The water-atomized powder was classified to provide a desired particle size distribution and a polymeric binder was added before consolidation by metal injection molding (MIM). The as-consolidated MIM product form was heated to an intermediate temperature to remove the binder. The MIM product form contained a globular MnS secondary phase when the microstructure was examined in both the transverse and longitudinal orientation.

### Example 4

Sulfur was not intentionally added to a vacuum induction melt of Fe—Mn alloy containing 28% manganese, 0.2% niobium, 0.08% carbon, balance iron. Ingots were homogenized, hot worked, and descaled. Rectangular pieces were cut from the ingot, cleaned, dimensions were measured, specimens were weighed, and corrosion testing was performed in Hank's Balanced Salt solution with added sodium bicarbonate at 37° C. at a pH of 7.4±0.2 for 14-15 days. Specimens were re-weighed and a corrosion rate calculation of 1.3928 milligrams/square inch/day was obtained.

### Example 5

Sulfur was added to a vacuum induction melt of Fe—Mn alloy containing 28% manganese, 0.2% niobium, 0.08% carbon, balance iron. Sulfur content measured in the solidified ingot was 400 ppm sulfur. Ingots were homogenized, hot worked, and descaled. Rectangular pieces were cut from the ingot, cleaned, dimensions were measured, specimens were weighed, and corrosion testing was performed in Hank's Balanced Salt solution with added sodium bicarbonate at 37° C. at a pH of 7.4±0.2 for 14-15 days. Specimens were re-weighed and a corrosion rate calculation of 3.8142 milligrams/square inch/day was obtained.

### Example 6

Sulfur was added to a vacuum induction melt of Fe—Mn alloy containing 28% manganese, 0.2% niobium, 0.08% carbon, balance iron. Sulfur content measured in the solidified ingot was 520 ppm sulfur. Ingots were homogenized, hot worked, and descaled. Rectangular pieces were cut from the ingot, cleaned, dimensions were measured, specimens were weighed, and corrosion testing was performed in Hank's Balanced Salt solution with added sodium bicarbonate at 37° C. at a pH of 7.4±0.2 for 14-15 days. Specimens were re-weighed and a corrosion rate calculation of 6.7569 milligrams/square inch/day was obtained.

### Example 7

We evaluated the corrosion rates with the addition of 400 parts per million (ppm) and 520 ppm sulfur to a biodegradable alloy of iron and 28% manganese. The corrosion rates were compared to the same alloy without added sulfur.

Over a two week period, the corrosion rate increased 2.9 times for the sample with 400 ppm added sulfur and 4.8 times for the sample with 520 ppm of added sulfur.

Iron(II) sulfide (FeS) converts spontaneously to MnS within the melt with a change in Gibbs free energy of Δ_{f} G=−118.0 kJ K¹ mol⁻¹ (kilojoules per degree kelvin·mole). We studied the effect on corrosion rates with the intentional addition of FeS to a biodegradable steel containing 28% manganese to form MnS precipitates within the steel structure.

Methods: Ingots of Bio4 biodegradable steel (28% Mn, 0.2% Nb, 0.08% C, balance iron) were melted with the addition of 500 and 2,500 ppm added sulfur as FeS. The ingots were melted, homogenized and hot worked (both hot forging and hot rolling). Samples of the ingots were compared to slices from a Bio4 ingot without added FeS. The sulfur level was measured in the final ingots.

Sample fabrication: Ingots were induction melted under vacuum with a 250 micron partial pressure of argon. The sulfide was added as FeS to prevent loss of the sulfide during melting. Ingots were homogenized under vacuum, and hot worked by forging and hot rolling. Samples of each hot worked ingot were prepared for corrosion testing by cutting rectangular pieces from slices using a diamond metallurgical saw, dressing by sanding with 2400 grit paper and electropolished to create a smooth surface. The samples were measured to the nearest 0.001 inches and the surface area calculated from the measurements. Samples were weighed to the nearest 0.1 mg.

Corrosion testing: Samples were immersed in Hank's Balanced Salt solution with added sodium bicarbonate (Sigma H9269-1L) at 37° C. and a pH of 7.4±0.2 for 14-15 days. The pH was maintained by adjusting the CO₂ concentration in the head space above the solution.

Samples were measured and weighed prior to being placed in the test solution and reweighed at the end of the test. Corrosion product was removed in distilled water under ultrasonic agitation for 1 minute, followed by multiple treatments of 10% W/V citric acid in an ultrasonic bath for 1 minute each. Samples were rinsed in distilled water, dried and weighed after each treatment cycle. The corrosion removal end point was determined by a change in slope of the plot of weight loss vs. treatment, as specified in paragraph 7.1.2.1-7.1.2.2 of ASTM G1-03 (Reapproved 2017).

Analysis: The surface area of each sample was calculated. The corrosion rate was then calculated as the loss in milligrams per square inch per day of exposure to Hank's solution.

Results: The target levels of added sulfur were 500 and 2500 ppm, however, the final ingots only contained 400 and 520 ppm respectively. The remaining added sulfur was lost to skull remaining in the melt crucible. Table 2 depicts the surface area, the weight loss in grams, the exposure in days and the calculated specific loss as milligrams loss per square inch per day.

TABLE 2

| | | Total | | Loss per Sq. |
|---|---|---|---|---|
| Sample | Surface | Loss | Days of | In. Per day |
| ID | Area | grams | exposure | in milligrams |
| Bio4 Control | 0.753868 | 0.0147 g | 14 | 1.392817 |
| (no added Sulfur) | Sq. In. | | | |
| Bio4 + | 0.089140 | 0.0051 g | 15 | 3.814225 |
| 400 ppm sulfur | Sq. In. | | | |
| Bio4 + | 0.068078 | 0.0069 g | 15 | 6.756955 |
| 520 ppm sulfur | Sq. In. | | | |

The corrosion rate as measured by loss per square inch of exposure per day of exposure was increased 2.9 times for the sample with 400 ppm sulfur and 4.8 times for the 520 ppm sulfur level.

Discussion: In this experiment, we added FeS to a steel charge of 28% manganese, 0.2% niobium, 0.08% carbon and the balance iron to form MnS precipitates in the final steel alloy. FeS converts spontaneously to MnS in the furnace with a change in Gibbs free energy of Δ_{f} G=−118.0 kJ K⁻¹ mol⁻¹. The target levels of added sulfur were 0.05% (500 ppm) and 0.25% (2500 ppm). The final measurements in the alloy were 400 ppm and 520 ppm. The remaining part of the charge was lost to the melt crucible as skull remaining stuck to the crucible, which was verified by analysis of the skull. The measurements of 400 and 520 ppm may be slightly low as the highest standard available in the laboratory was 270 ppm.

Corrosion is a surface area phenomenon, particularly with variants of Bio4 steel which is fabricated to prevent corrosion from progressing down grain boundaries beyond the current surface layer of grains. The current experiment was initiated to show that the corrosion rates could be increased by forming features in the surface that both increase the local susceptibility to corrosion and add additional pseudo corrosion surface area to an implant's surface in the form of the surface area that surrounds a reactive inclusion. The current example contained inclusions approximately shaped as 2 micron by 4 micron ovoid solids.

Conclusion: As has been seen in other experiments provided herein, adding a sulfur components to a manganese rich alloy increases the corrosion rate in a controllable fashion.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A biodegradable alloy suitable for use in a medical implant, comprising at least 50% iron by weight, at least 25% manganese by weight, and at least 0.01% sulfur and/or selenium by weight, wherein the biodegradable alloy is nonmagnetic.

2. The biodegradable alloy of claim 1, substantially free of chromium.

3. The biodegradable alloy of claim 1, substantially free of nickel.

4. The biodegradable alloy of claim 1, wherein sulfur and manganese form a manganese sulfide secondary phase.

5. The biodegradable alloy of claim 1, wherein selenium and manganese form a manganese selenide secondary phase.

6. The biodegradable alloy of claim 1, wherein the sulfur or selenium is dispersed equally in the biodegradable alloy.

7. The biodegradable alloy of claim 1, comprising at least 60% iron by weight.

8. The biodegradable alloy of claim 1, comprising at least 30% manganese by weight.

9. The biodegradable alloy of claim 1, in the form of a wrought product, a cast product, or a powder metallurgy product.

10. The biodegradable alloy of claim 1, having a degradation rate of about 0.155 to 3.1 mg/cm² under physiological conditions.

11. The biodegradable alloy of claim 1, comprising 0.01% to 0.35% sulfur and/or selenium by weight.

12. The biodegradable alloy of claim 11, comprising 0.01% to 0.20% sulfur and/or selenium by weight.

13. The biodegradable alloy of claim 11, comprising 0.02% to 0.10% sulfur and/or selenium by weight.

14. A method of producing a biodegradable alloy with a desirable degradation rate, the method comprising:
(a) adding a composition comprising sulfur and/or selenium to a molten mixture to produce the biodegradable alloy, wherein the molten mixture has at least 50% iron by weight and at least 25% manganese by weight, and wherein the biodegradable alloy comprises at least 0.01% sulfur and/or selenium by weight, and
(b) cooling the biodegradable alloy.

15. (canceled)

16. (canceled)

17. The method of claim 14, wherein the sulfur and/or selenium is added at 100 to 3500 parts per million.

18. The method of claim 14, wherein the composition comprising sulfur is iron(II) sulfide.

19. (canceled)

20. (canceled)

21. (canceled)

22. (canceled)

23. (canceled)

24. (canceled)

25. (canceled)

26. (canceled)

27. The method of claim 14, wherein the biodegradable alloy is cooled at a rate of 30° C./min to 60° C./min.

28. (canceled)

29. (canceled)

30. (canceled)

31. An implantable medical device comprising a biodegradable alloy of claim 1.

32. The implantable medical device of claim 31, wherein the implantable medical device is selected from the group consisting of a bone screw, a bone anchor, a tissue staple, a craniomaxillofacial reconstruction plate, a surgical mesh, a fastener, a reconstructive dental implant, and a stent.

33. A method of producing a biodegradable alloy with a desirable degradation rate, the method comprising adding 100 to 3500 parts per million sulfur to a molten mixture having at least 50% iron by weight and at least 25% manganese by weight, thereby producing a biodegradable alloy having at least 0.01% sulfur by weight.
